(19) United States
(12) Patent Application Publication
(10) Pub. No.: US 2015/0232954 A1
(43) Pub. Date: Aug. 20, 2015

(54) Method for quantifying different viral forms of the DNA of the HIV virus

(71) Applicants: UNIVERSITE PARIS DIDEROT - PARIS 7, Paris (FR); CENTRE NATIONAL DE LA RECHERCHE SCIENTIFIQUE, Paris (FR); ECOLE NORMALE SUPERIEURE DE CACHAN, Cachan Cedex (FR)

(72) Inventors: Soundasse Munir, Pontoise (FR); Sylvain Thierry, Paris (FR); Frédéric Subra, Paris (FR); Eric Deprez, Boissettes (FR); Olivier Delelis, Viroflay (FR)

(21) Appl. No.: 14/406,767

(22) PCT Filed: Jun. 12, 2013
(86) PCT No.: PCT/IB2013/054809

§ 371 Date: Dec. 10, 2014

(30) Foreign Application Priority Data
Jun. 12, 2012 (FR) 12 55489

(51) Int. Cl. C12Q 1/70

(52) U.S. Cl. CPC C12Q 1/703; C12Q 2600/136; C12Q 2600/158

(57) ABSTRACT

The invention relates to a method for quantifying linear forms of the DNA genome of the human immunodeficiency virus 1 (HIV-1) in a sample, wherein: a ligation reaction is performed with a double-stranded oligonucleotide suitable for ligation with the linear form of the HIV-1 DNA genome having a mature 3′ end and/or the linear form of the HIV-1 DNA genome having a non-mature 3′ end; a quantitative PCR is carried out from the ligation product obtained in the previous step; and the quantity of the linear form of the HIV-1 DNA genome having a mature 3′ end and/or the quantity of the linear form of the HIV-1 DNA genome having a non-mature 3′ end in the sample are deduced therefrom.

### FIELD OF THE INVENTION

The present invention relates to a method of quantifying different forms which the viral DNA of the human immunodeficiency virus (HIV) can adopt, especially the linear, mature or non-mature forms, and the circular form 1-LTR.

### PRIOR ART

The human immunodeficiency virus (HIV) is part of the Retrovirus family. During infection, the viral RNA is converted into a double-stranded linear viral DNA molecule. The linear DNA resulting from the reverse transcription then undergoes a maturation step at its LTR (Long Terminal Repeat) ends. This step, likewise called 3′-processing, consists in an endonucleolytic attack which suppresses two bases, at each 3′ end of the virus (Delelis et al. (2008) Retrovirology 5:114). The maturation step is indispensable to make the DNA ends compatible with the integration reaction. This integration step of the DNA viral genome is described in the literature as being an essential step of the replication cycle (Brown et al. (1990). Curr. Top. Microbiol. Immunol. 157:19-48). In fact, the integration of the virus, ensured by the viral enzyme named integrase, ensures the retention of the virus information within the cell, but also the production of new infectious viral particles and thus the propagation of the virus. It should be noted that the viral DNA can also be found in circular forms (with 1 or 2 LTR) in the cell but that these viral forms are considered as not having any key function in the replication cycle and viral progression (Sloan & Wainberg (2011) Retrovirology 8:52 and Wu (2008) Retrovirology 5:61). They are considered as “dead-end” molecules.

As a result, the quantification of the linear viral DNA, the only viral form capable of the integration of the virus, proves to be a major issue. Within this framework, several techniques based on the PCR (Polymerase Chain Reaction) exist for quantifying the different forms of viral DNA: integrated form, circular viral DNA with 2 LTR and “total” viral DNA (Graf et al. (2011) Plos. Pathog. 7:e1001300). The total viral DNA represents the totality of all the viral DNAs present in the cell. PCR methods have been developed for detecting linear viral DNA but do not allow it to be quantified (Pearson et al. (2002) Journal of Virology 17:8518-8531; Mohammed et al. (2011) Journal of Virology 85:4654-66). Moreover, the Southern blot technique allows all of the viral DNA forms: circular or linear, to be visualized (Zennou et al. (2000) Cell 101:173-85), but this technique is not quantitative and is lengthy, not very sensitive and necessitates radioactive material.

Thus, at the present time, no reliable and reproducible technique is available to quantify linear viral DNA and even less to quantify immature linear viral DNA from mature viral DNA (that is to say having been subjected to the 3′-processing reaction). It must be pointed out that the calculation method (quantity of linear viral DNA deduced by subtraction of the quantity of “total” viral DNA from the 2-LTR viral DNA, integrated and 1-LTR) is not possible since a highly represented form (the 1-LTR rings) is not quantifiable to date. In addition, these techniques necessitate a large quantity of viral material.

### DESCRIPTION OF THE INVENTION

The present invention follows from the perfection, by the inventors, of a method, based on LM-PCR (Linker-mediated PCR), of the linear forms and of the circular form with 1-LTR of the DNA genome of HIV-1.

LM-PCR, well known to the person skilled in the art, is described in particular by Mueller & Wold (1989) Science 246:780-786 and Pfeifer et al (1989) Science 246:810-813.

Thus, the present invention relates to a method of quantification of linear forms of the DNA genome of the human immunodeficiency virus 1 (HIV-1) in a sample, in which:
a ligation reaction is carried out with a double-stranded oligonucleotide adapted for a ligation with the 3′ mature linear form and/or the 3′ non-mature linear form of the DNA genome of HIV-1,
a quantitative PCR is carried out starting from the ligation product obtained in the preceding step,
the quantity of the 3′ mature linear form and/or of the 3′ non-mature linear form of the DNA genome of HIV-1 in the sample is deduced therefrom.

The genome of the HIV-1 can be RNA, as in the virion. It can likewise be DNA, in integrated form, in the genome of the host, or in non-integrated form. In this last case, it can then be present in linear form or in circular form. The circular forms are called 1-LTR or 2-LTR, depending on whether they have a single LTR (Long Terminal Repeat) or two. The linear forms, for their part, can be mature or non-mature at the 3′ end.

The PCR (Polymerase Chain Reaction) is well-known to the person skilled in the art. As it is understood here, a quantitative PCR represents a polymerization chain reaction allowing the quantity of nucleic acid to be amplified initially present in the sample having been subjected to the PCR to be determined. Numerous types of quantitative PCR are known to the person skilled in the art.

As will appear clearly to the person skilled in the art, a “quantification” and a “quantity” according to the invention are absolute, and non-relative, that is to say that they provide access to a number of molecules, to a number of moles, or to a mass, as well as to the corresponding concentrations or percentages.

As it is understood here, the double-stranded oligonucleotide can likewise be called linking oligonucleotide or “linker”; it is formed from the association by hybridization of two oligonucleotides of which at least one part of their sequences are complementary.

The ligation reaction is well-known to the person skilled in the art; it consists of creating a covalent link between the 3′ end of one nucleic acid and the 5′ end of another nucleic acid. It is generally implemented with the aid of a ligase.

Preferably, the linker defined above allows a ligation with the 3′ mature linear form of the DNA genome of HIV-1 and comprises a sticky end of two nucleotides of sequence 5′-GT-3′, and the total quantity of the linear forms of the genome of the HIV-1 in the sample is deduced from the quantitative PCR. More preferably, the linking oligonucleotide is then formed from the association of a 5′-GCGGTGACCCGGGAGATCTGAATTC-3′ sequence (SEQ ID NO: 5), or a sequence having at least 90% identity with SEQ ID NO: 5, and from a sequence 5′-GTGAATTCAGATC-3 (SEQ ID NO: 6), or a sequence having at least 90% identity with SEQ ID NO: 6.

As is understood here, a double stranded nucleic acid is said to have a sticky end when at least one of the ends of the nucleic acid has a protruding strand which cannot pair with the other strand. Conversely, a double-stranded nucleic acid is said to have a blunt end when the two strands are completely paired at at least one of the ends of the nucleic acid.

A sequence having at least 90% identity with SEQ ID NO: X according to the invention, preferably has at least 95% identity with SEQ ID NO: X. This sequence differs especially from SEQ ID NO: X by the insertion, the suppression or the substitution of at least one nucleotide. As is understood here, the percentage of identity between two sequences is defined as the number of positions for which the bases are identical when the sequences are aligned in optimal manner, divided by the total number of bases of the larger of the two sequences. Two sequences are stated to be aligned in optimal manner when the percentage of identity is maximal. Furthermore, as will appear clearly to the person skilled in the art, it can be necessary to use some additions of incomplete positions (of “gaps”) so as to obtain an optimal alignment between the two sequences. Moreover, the sequence displaying at least 90% identity with SEQ ID NO: X according to the invention preserves the function, especially of primer or of probe, of SEQ ID NO: X, that is to say it allows an amplification or a detection of the nucleic acid to be amplified.

Likewise preferably, the linking oligonucleotide defined above allows a ligation specifically with the 3′ non-mature linear form of the genome of HIV-1 and comprises a free end, and the quantity of the non-mature linear form of the genome of the HIV-1 in the sample is deduced from the quantitative PCR. More preferably, the linking oligonucleotide is then formed from the association of a sequence 5′-GCGGTGACCCGGGAGATCTGAATTC-3′ (SEQ ID NO: 5), or a sequence having at least 90% identity with SEQ ID NO: 5, and of a sequence 5′-GAATTCAGATC-3′ (SEQ ID NO: 7), or a sequence having at least 90% identity with SEQ ID NO: 7.

Preferably, the quantity of the mature linear form of the genome of the HIV-1 in the sample is determined by subtraction of (i) the quantity of the non-mature linear form of the genome of the HIV-1 in the sample determined according to the invention from (ii) the total quantity of the linear forms of the genome of the HIV-1 in the sample determined according to the invention.

Likewise preferably, the quantitative PCR according to the invention is a real-time PCR, more preferably conducted with probes of the hybridization probe type.

The real-time PCR is well-known to the person skilled in the art. Briefly, real-time PCR combines the amplification of a nucleic acid and the detection by fluorescence of the nucleic acid amplified. A conventional PCR is generally carried out in the presence of probes, especially of hybridization probes, that is to say probes which hybridize with the nucleic acid amplified and which emit a fluorescent signal in response to this hybridization. The emission of fluorescence is measured as a function of the PCR cycles. The PCR cycle for which the fluorescence signal emitted is measured above a threshold level, generally above the basal level or the background fluorescence level, is called the threshold cycle (Ct). It has been shown that Ct is proportional to the decimal logarithm of the quantity of nucleic acid to be amplified initially present in the PCR reaction (see, for example, “Real-time PCR” in Advanced Methods S., Dorak M T. ed, Taylor and Francis, Oxford 2006).

Among the numerous hybridization probes according to the invention known to the person skilled in the art applicable according to the invention, it is especially possible to cite the probes of the Molecular Beacon type which are described, for example, in the international application WO 98/10096.

In a preferred manner, the quantitative PCR according to the invention is carried out in two steps: a first amplification step without quantification of the nucleic acid amplified and a second amplification step with quantification of the nucleic acid amplified.

In a more preferred manner, the first step of the quantitative PCR according to the invention is carried out with a primer comprising a sequence 5′-GCGGTGACCCGGGAGATCTGAATTC-3′ (SEQ ID NO: 5), such as a sequence 5′-GCGCGCGGCGGTGACCCGGGAGATCTGAATTC-3′ (SEQ ID NO: 25), or a sequence having at least 90% identity with SEQ ID NO: 5 or 25, and with a primer comprising a sequence 5′-CTCGCCTCTTGCCGTGCGCG-3′ (SEQ ID NO: 9), or of a sequence having at least 90% identity with SEQ ID NO: 9.

In a more preferred manner, likewise the second step of the quantitative PCR according to the invention is carried out with a primer comprising a sequence 5′-GCGGTGACCCGGGAGATCTGAATTC-3′ (SEQ ID NO: 5), or a sequence having at least 90% identity with SEQ ID NO: 5, and a primer comprising a sequence 5′-GAGTCCTGCGTCGAGAGATC-3′ (SEQ ID NO: 26), or a sequence having at least 90% identity with SEQ ID NO: 26, and a hybridization probe comprising a sequence 5′-CCCTCAGACCCTTTTAGTCAGTGTGGAA-3′ (SEQ ID NO: 27), or a sequence having at least 90% identify with SEQ ID NO: 27, and/or a hybridization probe comprising a sequence 5′-TCTCTAGCAGTGGCGCCCGAACAG-3′ (SEQ ID NO: 28), or a sequence having at least 90% identity with SEQ ID NO: 28.

Furthermore, in the method defined above, it is preferred that the first step of the quantitative PCR comprises from 9 to 30 cycles, more preferably 10 to 14 cycles, and even more preferably approximately 12 cycles.

The deduction step of the quantity of the mature 3′ linear form and/or of the non-mature 3′ linear form of the DNA genome of the HIV-1 in the sample can especially be implemented with reference to a calibration curve. This calibration curve can especially be obtained by carrying out the two first steps of the quantification procedure according to the invention starting from known quantities of double-stranded DNA molecules mimicking the mature 3′ linear form and/or the non-mature 3′ linear form of the DNA genome of the HIV-1. These known quantities of DNA molecules mimicking the mature 3′ linear form and/or the non-mature 3′ linear form of the DNA genome of the HIV-1 are preferably obtained by digestion of known quantities of a plasmid with the aid of restriction enzymes especially allowing double-stranded DNA molecules having at least one blunt end, for the molecules mimicking the linear non-mature form, or at least one sticky end of 2 nucleotides, for the molecules mimicking the non-mature linear form to be released. Furthermore, it is preferred that the double-stranded DNA molecules mimicking the 3′ mature linear form and/or the 3′ non-mature linear form of the DNA genome of the HIV-1 essentially have the same size and/or essentially the same sequence as the latter. However, limited variations of sizes or of sequences are acceptable. In particular, the sticky end of 2 nucleotides of the double-stranded DNA molecule mimicking the mature linear form of the DNA genome of the HIV-1 can have a sequence different from that of the mature linear form; in this case, the person skilled in the art will understand that it is appropriate as a result to modify the sticky end of the linking oligonucleotide adapted for ligation with this double-stranded DNA and used in the preparation of the calibration curve, for which it is complementary to that of the double-stranded DNA.

Furthermore, the invention likewise concerns a method of quantification of the 1-LTR circular form of the DNA genome of the HIV-1 in a sample, in which a quantitative PCR according to the invention is carried out with the aid of two primers respectively comprising a sequence 5′-GCGCTTCAGCAAGCCGAGTCCT-3′ (SEQ ID NO: 1), or a sequence having at least 90% identity with SEQ ID NO: 1, and a sequence 5′-GTCACACCTCAGGTACCTTTAAGACCAATGAC-3′ (SEQ ID NO: 2), or a sequence having at least 90% identity with SEQ ID NO: 2.

Preferably, quantitative PCR is carried out with a hybridization probe comprising the sequence 5′-CACAACAGACGGGCACACACTACTTGA-3′ (SEQ ID NO: 3), or a sequence having at least 90% identity with SEQ ID NO: 3 and/or with a hybridization probe comprising the sequence 5′-CACTCAAGGCAAGCTTTATTGAGGC-3′ (SEQ ID NO: 4), or a sequence having at least 90% identity with SEQ ID NO: 4.

Preferably likewise, the quantitative PCR employed in the quantification method of the circular form with 1-LTR of the DNA genome of the HIV-1 according to the invention requires the definition of a precise duration of the elongation step of the PCR. Thus, it is preferred that the elongation step, that is to say the step conducted at a temperature favoring the activity of the thermostable DNA polymerase, has a duration of 20 to 30 seconds, more preferably of 23 to 27 seconds, and even more preferably of approximately 25 seconds.

The invention likewise concerns a method of screening for compounds capable of modifying the quantity of a non-integrated form of the genome of the HIV-1, comprising the implementation of a method of quantification of the linear forms or of the circular form with 1-LTR such as defined above. In a preferred mode of implementation of the screening method according to the invention, the compounds are capable of inhibiting the maturation of the linear form of the DNA genome of the HIV-1 and a method of quantification of the linear forms such as defined above is implemented.

The invention will be further explained with the aid of the Examples and Figures which follow.

### DESCRIPTION OF THE FIGURES

FIGS. 1A, 1B and 1C

FIG. 1A: PCR on dilutions of the plasmid pLIN-HIV-ScaI. Successive dilutions of plasmid pLIN-HIV-ScaI were carried out (from 2^{e}6 copies to 2^{e}0 copies). The calculated efficacy of the PCR with the aid of the method of the second derivative provided by the Light Cycler quantification program, version 3.5 (Roche Diagnostics) is 90%. The fluorescence intensity (ordinate axis) is tracked based on the number of cycles (abscissa axis).

FIG. 1B: Results of the different PCRs using the linkers and the different substrates studied.

FIG. 1C: Photograph of an electrophoresis gel in which the amplification products have been caused to migrate.

FIGS. 2A, 2B, 2C and 2D

FIG. 2A: Amplification scheme using the primers for the quantification of the 1-LTR bands on the substrates 1-LTR (1), 2-LTR (2) and on the linear viral DNA (3).

FIG. 2B: Plasmids used: Plasmid 1-LTR (1), 2-LTR (2) and DNA mimicking linear viral DNA after digestion of the plasmid 2-LTR by the enzyme ScaI (3).

FIG. 2C: Efficacy of the quantitative 1-LTR PCR. Successive dilutions of the plasmid 1-LTR (from 2^{e}6 copies to 2^{e}2 copies) were carried out. The fluorescence intensity (ordinate axis) is followed as a function of the number of cycles (abscissa axis).

FIG. 2D: Photograph of an electrophoresis gel in which the amplification products obtained starting from the plasmid 1-LTR (1), of the plasmid 2-LTR (2) and of the DNA mimicking the linear viral DNA after digestion of the plasmid 2-LTR by the enzyme ScaI (3) have been caused to migrate.

FIGS. 3A, 3B, 3C, 3D, 3E and 3F

Kinetics of different forms of viral DNA after infection of MT4 cells by the WT virus (in the presence or not of RAL) or by a virus D116N. The condition WT without raltegravir is symbolized by black squares. The condition WT with raltegravir is symbolized by black diamonds. The condition D116N is symbolized by white rings. The number of copies of viral DNA, normalized by the quantity of total cellular DNA (ordinate axis) is given as a function of the time in hours (abscissa axis).

FIG. 3A: kinetics of the total viral DNA.

FIG. 3B: kinetics of the 2-LTR bands.

FIG. 3C: kinetics of the 1-LTR bands.

FIG. 3D: kinetics of the viral DNA integrated.

FIG. 3E: percentage of 2-LTR bands (quantity of 2-LTR bands in total viral DNA, ordinate axis) as a function of time (abscissa axis, in hours) for the condition WT without Raltegravir (WT, black bars), the condition WT with Raltegravir (RAL, grey bars) and the condition D116N (white bars).

FIG. 3F: also the percentage of 1-LTR bands (quantity of 1-LTR bands in total viral DNA, ordinate axis) as a function of time (abscissa axis, in hours) for the condition WT without Raltegravir (WT, black bars), the condition WT with Raltegravir (RAL, grey bars) and the condition D116N (white bars).

FIGS. 4A, 4B, 4C and 4D

FIGS. 4A, 4B and 4C: Kinetics of the mature and non-mature linear viral DNA after infection of CEM cells by the WT virus (in the presence or not of Raltegravir, FIG. 4B and FIG. 4A respectively) or by the D116N virus (FIG. 4C). The quantity of non-mature DNA is symbolized by a white circle. The quantity of mature DNA is symbolized by a black square. The number of copies of viral DNA, normalized by the quantity of total cellular DNA (ordinate axis), is given as a function of the time in hours (abscissa axis).

FIG. 4D: The percentage of mature DNA (quantity of mature DNA divided by the quantity of total linear DNA) is represented for the three conditions: WT (black), WT in the presence of Raltegravir (grey) and D116N (in white).

FIG. 5

The CEM cells were infected by the pNL4.3 virus in the absence (WT) or in the presence of 20 μM of FZ41 (WT+FZ41). The mature and non-mature viral DNA is quantified 24 hours after infection. The proportion of each species of mature linear viral DNA (grey) and non-mature (white) is represented.

### EXAMPLES

### Example 1

### Materials and Methods

Cells and virus. The lymphoid cell line CEM (NIH, catalogue reference 117) was cultured in RPMI1640 medium (Invitrogen, Cat #61870-044) supplemented by 10% of fetal calf serum (SVF) (PAA, Lot #A10109-113). The 293T and HeLa cells were cultured in DMEM (Invitrogen, Cat #31966-047) (Dulbecco's modified Eagle medium) supplemented by 10% of SVF. The stocks of HIV-1 were prepared by transfection of 293T cells with the different molecular clones derived from pNL4-3 (Gelderblom et al. (2008) Retrovirology 9;5:60). The viruses Δenv NLENG1-ES-IRES WT and NLENG1-ES-IRES D116N (Gelderblom et al. (2008) Retrovirology 9;5:60) respectively code the wild-type and the catalytically inactive mutant D116N of integrase. The element IRES is necessary for the suppression of the GFP. The pseudotyping of the virus Δenv was carried out by co-transfection of 293T cells with a VSV-G plasmid (Addgene, plasmid 8454) with the aid of the calcium phosphate method. The viral supernatants were filtered through a filter with pore diameter 0.45 μm (Sartorius, reference 16537K) and then frozen at −80° C. Viral infection. The content of p24^{gag} antigen of the HIV-1 of the culture supernatants was determined by ELISA (Perkin-Elmer Life Sciences, Paris, France reference: NEK050B001KT). 40 ng of p24^{gag} antigen per 10⁶ cells, which corresponds to a multiplicity of infection (m.o.i.) of 0.1, were used for infection. When necessary, the cells were treated in the presence of the integrase inhibitor raltegravir (RAL) to 500 nM. 2 to 5 million cells were collected at different times after infection. The cells were then washed in PBS, then centrifuged, and the dry cell pellets were frozen at −80° C. until use thereof. The total cell DNA of the infected cells of the cell line CEM was purified with the aid of the kit QIAamp DNA Blood mini kit (Qiagen, reference 51104) according to the instructions of the manufacturer. In order to degrade the residual transfection plasmids, the DNA extracted was incubated with 5 U of DpnI (NEB, reference R0176S) in a buffer comprising 20 mM Tris pH 7.9 for 4 hours at 37° C.

Plasmids. Four Plasmids Were Constructed: p1-LTR, p2-LTR, p-LIN-HIV-ScaI and p-LIN-HIV-NdeI.

p-LIN-HIV-ScaI was constructed as follows: amplification by PCR with the aid of the oligonucleotide 25tSca-HIV (5′-GCGGTGACCCGGGAGATCTGAATTCAGTACTGGAAGGGCTAATTTGGTCCC-3′, SEQ ID NO: 8) and of the oligonucleotide MS1 (5′-CTCGCCTCTTGCCGTGCGCG-3′, SEQ ID NO: 9) using the plasmid of pNL4.3 as matrix. The amplification product was then purified and introduced into the vector pGEMT-easy (Proméga, reference A1360). The nucleotides underlined represent the viral DNA sequence of the LTR5′ (region U3). It is to be pointed out that a ScaI site (5′-AGTACT-3′, SEQ ID NO: 10) exists in the position 315 in the plasmid pNL4.3. This ScaI site was modified at a site not allowing cutting by the restriction enzyme ScaI (5′-AGTACT-3′ mutated into 5′-AGAACT-3′, SEQ ID NO: 11) by a site-specific directed method of mutagenesis (QuikChange Lightning Kits, Agilent) with the aid of oligonucleotides of sequences 5′-CCCGAGAGCTGCATCCGGAGAACTACAAAGACTGCTGACATCG-3′, (SEQ ID NO: 12) and 5′-CGATGTCAGCAGTCTTTGTAGTTCTCCGGATGCAGCTCTCGGG-3′, SEQ ID NO: 13). The digestion by ScaI and AatII leads to a fragment mimicking the non-mature end of the viral DNA. After purification, this fragment is used for implementing a ligation reaction with the double strand liaison oligonucleotides (or “linker”) 25t/11b or 25t/11GT.

p-LIN-HIV-NdeI was constructed by site-specific directed mutagenesis (QuikChange Lightning Kits, Agilent) of the plasmid p-LIN-HIV-ScaI, by mutating the site ScaI (5′-AGTACT-3′, SEQ ID NO: 10) into a site NdeI (5′-CATATG-3′, SEQ ID NO: 14) at the end of the viral DNA with the aid of oligonucleotides of sequences 5′-CCGGGAGATCTGAATTCAGTCATATGGAAGGGCTAATTTGGTCC-3′, SEQ ID NO: 15) and 5′-GGACCAAATTAGCCCTTCCATATGACTGAATTCAGATCTCCCGG-3′, SEQ ID NO: 16). The digestion by NdeI and AatII (of which the digestion site is only present in the vector pGEMT-easy) leads to a fragment mimicking the mature 3′ end of the viral DNA. After purification, this fragment is used as a PCR standard aiming to quantify the mature DNA in 3′. It is important to note that the double digestion NdeI and AatII allows a fragment mimicking the mature DNA of HIV-1 (cohesive end of 2 pairs of bases (5′-TA-3′) to be obtained. However, the nature of these bases differs with those found at the end of the mature genome (5′-AC-3′). However, the nature of these bases does not interfere at all with the method of quantification of the mature viral DNA.

The primers 1LTR LA1 (5′-GCGCTTCAGCAAGCCGAGTCCT-3′, SEQ ID NO: 1) and 1LTR LA16 (5′-GTCACACCTCAGGTACCTTTAAGACCAATGAC-3′, SEQ ID NO: 2) were used in order to amplify by PCR the region env-LTR-gag contained in the circular molecules with 1-LTR starting from extracts of cells infected by HIV-1. The plasmid p1-LTR was then obtained by cloning the amplification product in the vector pGEMT-easy.

The plasmid p2-LTR was constructed as follows: the primers ScaI-LTR3′ (5′-CGGGAGATCTGAATTCAGTACTGCTAGAGATTTTCCACACTGACTAAAAG-3′, SEQ ID NO: 17) and 1LTR LA16 (5′-GTCACACCTCAGGTACCTTTAAGACCAATGAC-3′, SEQ ID NO: 2) were utilized in order to amplify the region env-LTR3′ and the amplification product was inserted into the vector pGEMT-easy in order to give the intermediate plasmid pSca-LTR3′. The site ScaI (5′-AGTACT-3′, SEQ ID NO: 10), present in the LTR3′ in position 10715 of the viral genome was mutated to 5′-AGCACT-3′, (SEQ ID NO: 18) by a site-specific directed mutagenesis method (QuikChange Lightning Kits, Agilent) with the aid of oligonucleotides of sequences 5′-AGCTGCATCCGGAGCACTTCAAGAACTGCT-3′(SEQ ID NO: 19) and 5′-AGCAGTTCTTGAAGTACTCCGGATGCAGCT-3′(SEQ ID NO: 20). The plasmid pSca-LTR3′and the plasmid pLIN-HIV-ScaI were digested by the enzyme ScaI. The fragments obtained, of size 2520 by and 1920 by were purified and ligated in order to obtain the plasmid p2-LTR. Finally, the site ScaI (5′-AGTACT-3′, SEQ ID NO: 10) present in the vector pGEMT-easy (in position 1890 of the vector) was mutated to a site 5′-AATACT-3′(SEQ ID NO: 21) with the aid of the oligonucleotides 5′-GTGACTGGTGAATACTCAACCAAGTCATTCTGAG-3′(SEQ ID NO: 22) and a 5′-CTCAGAATGACTTGGTTGAGTATTCACCAGTCAC-3′ (SEQ ID NO: 23), in order to allow the linearization of the plasmid p2-LTR by digestion of this by the enzyme ScaI.

Quantification of 3′ non-mature and mature linear forms of the DNA of HIV-1 by LM-PCR. The strategy used in order to quantify the linear viral DNA is based on the ligation of a linking oligonucleotide (or “linker”) at the ends of the viral ends followed by two PCRs according to the protocol of Pierson et al. (Pierson et al. (2002) Journal of Virology 17:8518-8531). Major improvements have been made in order to implement an approach of quantitative PCR type.

The linker formed of two oligonucleotides 25t and 11b (25t: 5′-GCGGTGACCCGGGAGATCTGAATTC-3′, SEQ ID NO: 5, 11b: 5′-GAATTCAGATC-3′, SEQ ID NO: 7) (linker 11b) and the linker formed of oligonucleotides 25t and 11GT (25t: 5′-GCGGTGACCCGGGAGATCTGAATTC-3′; SEQ ID NO: 5, 11GT: 5′-GTGAATTCAGATC-3′, SEQ ID NO: 6) (linker 11GTb), were used respectively for the quantification of the non-mature linear DNA of HIV-1 and of the mature linear DNA of HIV-1.

For the quantification of the non-mature linear viral DNA, the efficacy of the procedure of ligation-amplification was determined by the addition of the linking oligonucleotide (linker 11b) to dilutions in series of the digestion fragment of pLIN-HIV-ScaI obtained by digestion by the enzymes ScaI and AatII. The dilutions in series of this fragment form the range of calibration of the non-mature DNA.

For the quantification of the mature linear viral DNA, the efficacy of the procedure of ligation-amplification was determined by addition of the linking oligonucleotide (linker 11TAb) formed from the oligonucleotides 25t and 11TA (11TA: 5′-TAGAATTCAGATC-3′, SEQ ID NO: 24) to dilutions in series of fragments obtained by digestion of pLIN-HIV-NdeI by NdeI and AatII. The dilutions in series of this fragment form the calibration range of the mature DNA.

It is to be noted that during the infection of cells by the HIV-1 virus, the end of the mature linear viral DNA differs from the end of the calibration range, it is for this reason that the quantification of the mature viral DNA in infected cells is carried out with the aid of the linker 11GTb in place of the linker 11TAb. This difference has no influence on the quantification of the mature viral DNA. The quantification of the mature linear viral DNA is thus obtained by means of comparison with the calibration range.

The different calibration curves were produced in the presence of DNA of non-infected cells in order to verify that the efficacy of the ligation/amplification was not influenced by the capture of linking oligonucleotide by the DNA of non-infected cells.

The linking oligonucleotides were associated by heating each complementary strand (at the final concentration of 3 μM) to 95° C. for 5 min then by a slow cooling until reaching ambient temperature. The linking oligonucleotides were added at the final concentration of 30 nM to a quantity of 200 ng to 2 μg of DNA of infected cells in a final volume of 20 μL in the presence of the ligase of the Quick ligation kit 0.8 μL, or 300 units of enzymes (NEB, reference M2200S) for 2 hours at ambient temperature in a final volume of 20 μL. The ligated DNA products were then purified with the switch charge purification PCR kit (Life Technology, CS12000) according to the instructions of the manufacturer, then eluted in 20 μL and subjected to a real-time PCR.

The quantifications were employed by real-time PCR on a Light Cycler apparatus (Roche Diagnostics, Meylan, France). The quantifications, with respect to the range used, were calculated with the aid of the second derivative method supplied by the quantification software of the Light Cycler, version 3.5 (Roche Diagnostics).

In a first round of PCR, 1/10 of the DNA purified beforehand was amplified in a reaction mixture of 20 μL comprising 1× LightCycler FastStart DNA master Hybprobes (Roche, reference 12239272001), 4 mM MgCl₂, 300 nM of primer 32t 5′-GCGCGCGGCGGTGACCCGGGAGATCTGAATTC-3′(SEQ ID NO: 25) and MS1 5′-CTCGCCTCTTGCCGTGCGCG-3′ (SEQ ID NO: 9).

In order to remain in the exponential phase, only 12 cycles were carried out. Thus, the conditions of the first round of PCR were the following: a denaturation step of 8 min at 95° C. then 12 amplification cycles (95° C. for 10 s, 60° C. for 10 s, and 72° C. for 31 s). The second round of nested PCR was carried out on 1/100 of the product of the first round of PCR in a mixture comprising 1× LightCycler FastStart DNA master Hybprobes buffer, 4 mM MgCl₂, 300 nM of 25t primers (5′-GCGGTGACCCGGGAGATCTGAATTC-3′ SEQ ID NO: 5) and MS2 (5′-GAGTCCTGCGTCGAGAGATC-3′; SEQ ID NO: 26) and 200 nM of hybridization probes MHFL* (5′-CCCTCAGACCCTTTTAGTCAGTGTGGAA^{a}-3′ (SEQ ID NO: 27) and MHLC* (5′-TCTCTAGCAGTGGCGCCCGAACAG^{b}-3′(SEQ ID NO: 28) (a: fluorescein in 3′; b: LCred640 in 5′ and phosphorylated in 3′).

The number of copies of mature and non-mature linear DNA was determined by reference to the calibration curves prepared by amplifying quantities ranging from 10 to 10⁵ copies of corresponding digested fragments.

Quantification of the 1-LTR bands. The quantifications were carried out by real-time PCR on a Light Cycler apparatus (Roche Diagnostics, Meylan, France) with the aid of the second derivative method provided with the Light Cycler quantification software, version 3.5 (Roche Diagnostics). The number of DNA copies of the 1-LTR bands was determined by quantifying the viral molecules with the aid of primers which hybridize to the genes gag and env, as well as to hybridization probes. The reaction mixtures contain 1× LightCycler FastStart DNA master HybProbes buffer (Roche Diagnostics reference 12239272001), 4 mM MgCl₂, 300 nM of 1-LTR LA1 primers (5′-GCGCTTCAGCAAGCCGAGTCCT-3′, SEQ ID NO: 1) and 1-LTR LA16 (5′-GTCACACCTCAGGTACCTTTAAGACCAATGAC-3′, SEQ ID NO: 2) and 200 nM of LTR FL* hybridization probes (5′-CACAACAGACGGGCACACACTACTTGA^{a}-3′, SEQ ID NO: 3) and LTR LC*(5′-CACTCAAGGCAAGCTTTATTGAGGC^{b}-3′, SEQ ID NO: 4) (^{a}Fluorescein at the 3′ end; ^{b}LC red 640 at the 5′ end and phosphorylated at the 3′ end) in a final volume of 20 μl. The conditions of the PCR cycles for the quantification of the 1-LTR bands are the following: denaturation step for 8 min followed by 40 cycles (95° C. for 10 s, 60° C. for 10 s, and 72° C. for 25 s). The number of copies of 1-LTR bands was determined by reference to a calibration curve prepared by amplifying quantities ranging from 100 to 10⁵ copies of p1-LTR.

### Results

### Quantification of the Linear DNA of HIV-1

The quantification of the viral DNA was carried out by following a PCR approach implementing a ligation (ligation-mediated PCR, LM-PCR) providing major improvements to that of Pierson et al. (Pierson et al. (2002) Journal of Virology 17:8518-8531).

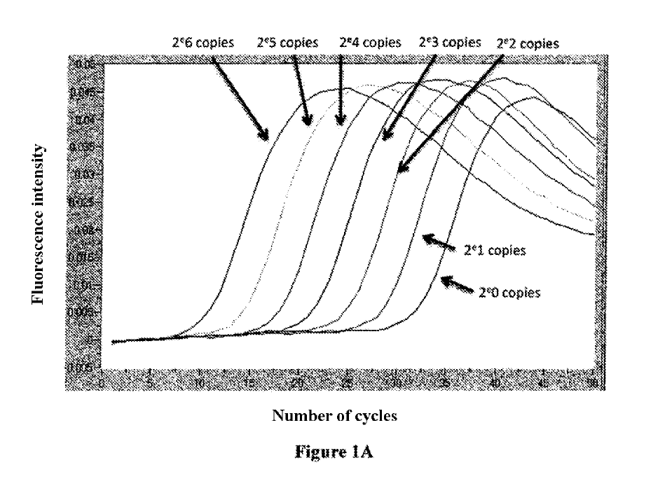

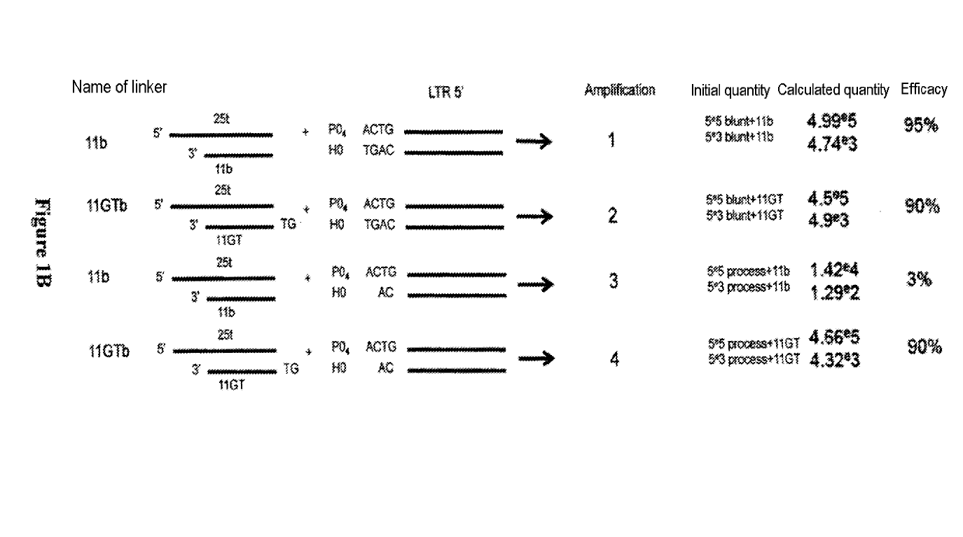

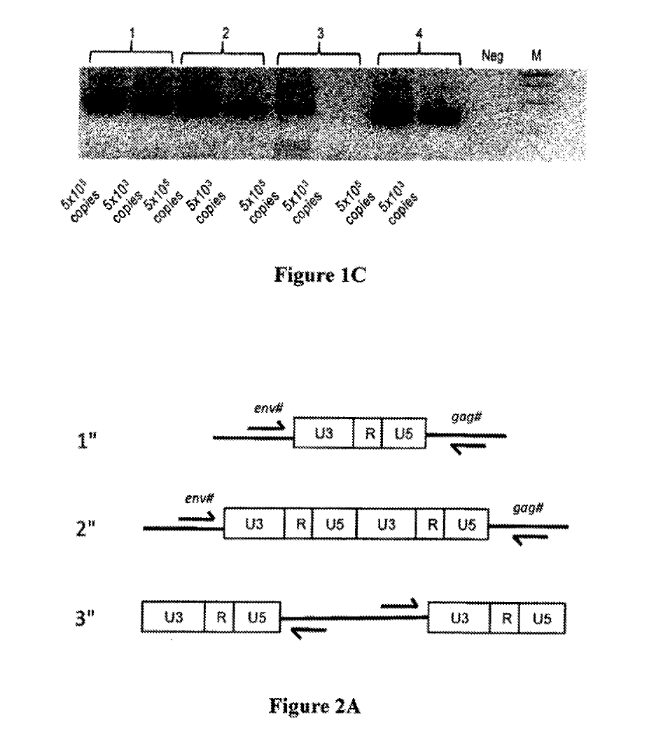

The efficacy and the sensitivity of the quantitative PCR with the primers 25t and MS2 are represented for pLIN-HIV-ScaI (FIG. 1A). The parameters used for pLIN-HIV-NdeI were identical to those determined for pLIN-HIV-ScaI indicating that the difference between the two plasmids concerning the end of the viral DNA (a ScaI site for pLIN-HIV-ScaI and an NdeI site for pLIN-HIV-ScaI) does not influence the quantitative PCR, as expected.

The DNA mimicking the non-mature end was obtained by digestion with ScaI/AatII then purification. The DNA mimicking the mature 3′ end was obtained by digestion with NdeI/AatII then purification. After purification, these fragments were quantified by quantitative PCR using the primers and the probes for the quantification of the total viral DNA (see Table 1). As described in FIG. 1B, the LM-PCR with the linker 11GTb leads to the detection of the 3′ mature and non-mature linear viral DNA with an efficacy of 90% and a sensitivity of 10 copies per 200,000 cells in the two cases (as indicated in the materials and methods section the efficacy was in fact determined with the aid of the linker 11TAb of which the sticky end of 2 nucleotides is complementary to that generated by the NdeI cut but for the sake of simplification it is the linker 11GTb which is represented in the figure).

The LM-PCR with the linking oligonucleotide (linker 11b) is only capable of detecting and of quantifying the non-mature viral DNA. As a result, the quantity of 3′ mature DNA is deducted by subtracting the quantity obtained with the linking oligonucleotide (linker 11GTb) from that obtained with the linking oligonucleotide (linker 11b). For each quantification (non-mature linear viral DNA and 3′ mature linear DNA), several dilutions of corresponding fragments were carried out in the DNA of non-infected cells (200 ng/μL). A linking oligonucleotide (11GTb linker for the non-mature linear DNA and linker 11b for the mature linear DNA) was ligated with each dilution of end of viral DNA (non-mature or 3′ mature). Consequently, the quantification obtained after PCR takes into account the efficacy of ligation. After ligation, the ligated DNA was purified with the aid of magnetic beads according to the instructions of the manufacturer (Invitrogen) in order to avoid the inhibition of the PCR due to mixing of the ligation reaction. The ligated DNA (1/10 of the ligation reaction) was then subjected to an amplification by real-time PCR with the primers 32t and MS1 (see Table 1).

The cycle number was determined empirically. In order to remain in the exponential phase, only 12 PCR cycles were carried out. Unexpectedly, decreasing the cycle number (for example 8 cycles) during the first PCR results in a poor amplification of the samples leading to an erroneous quantification. In the same way, increasing the cycle number does not improve the quantification. Moreover, after 30 cycles, the curves of the plasmid range used for the quantification (dilution of 10 in 10) are quasi-superimposed; consequently the quantification of the sample is impossible. Thus 12 cycles are sufficient to allow a quantification of all the DNA dilutions ligated at the end of the second PCR. The amplified products (1/100) were then subjected to the second PCR. The efficacy of each quantification (3′ non-mature and mature) is described in FIGS. 1B and 1C.

As indicated above, it is important to note that the end of the fragment resulting from the digestion by NdeI is similar but not identical to that found at the 3′ end in the infected cells (5′-ATTG and not 5′-ACTG in the infected cells), which makes necessary the use of the linking oligonucleotide 11TAb for these experiments in the place of the linking oligonucleotide 11GTb. As described by Pierson et al. (Pierson et al. (2002) Journal of Virology 17:8518-8531), the linking oligonucleotide (linker 11GTb) is capable of detecting the non-mature viral end. The reason may be due to the fact that the sticky end of the linking oligonucleotide (5′-GT) is not involved in the ligation with the phosphate of the 5′ side of the genome. However, by using this linking oligonucleotide (linker 11GTb), both the non-mature end and the 3′ mature end can be efficiently detected. Overall, the inventors have shown that asymmetric linking oligonucleotides can be used for an efficient quantification of the ends of the viral DNA.

Consequently, the pairs 25t/11GT and 25t/11b will be used both to quantify the non-mature and 3′ mature ends during the natural course of the infection.

Quantification of the 1-LTR bands

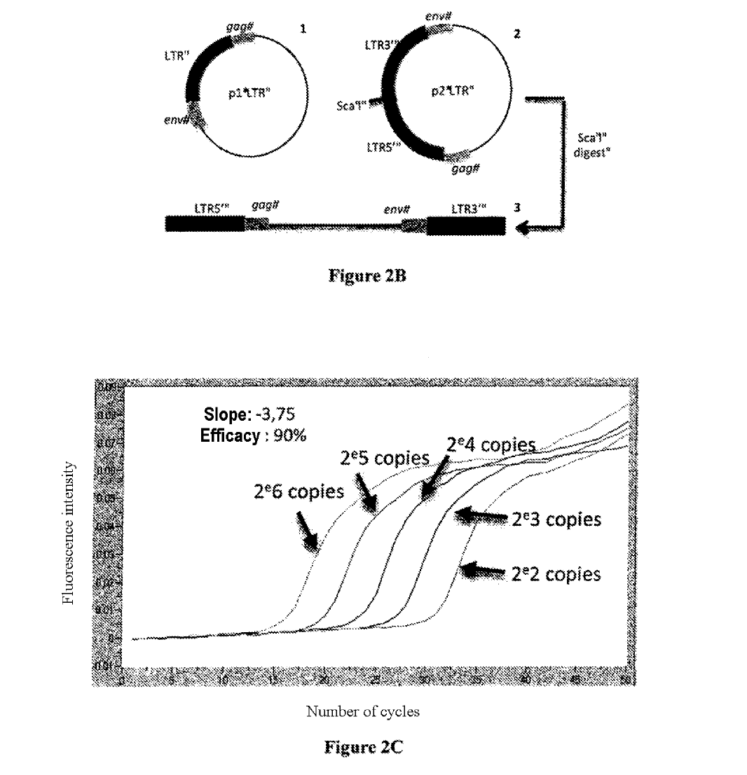

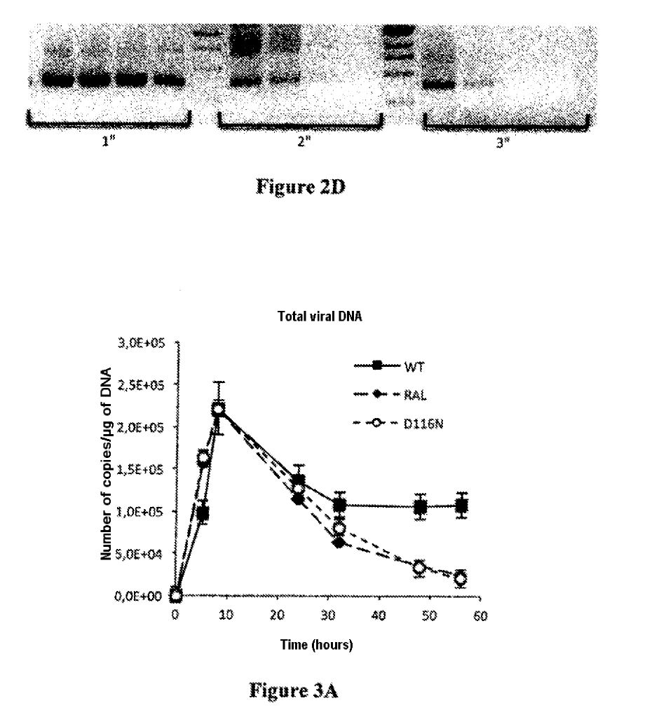

Several methods based on the PCR have been proposed in order to quantify the 1-LTR bands (Bukrinsky et al. (1992) Proc Natl Acad Sci USA 89, 14:6580-4). These PCRs are based on the hybridization of primers in the genes env and gag (FIG. 2A1). However, it has been reported that the methods based on the PCR do not allow a correct quantification of the 1-LTR bands (Yoder & Fishel (2006) Journal of Virological Methods 138, 1-2:201-6). In fact, as is shown in FIG. 2A2, the primers used for the 1-LTR bands could lead to an amplification of the LTR-LTR region present in the 2-LTR bands.

In addition, as described by Yoder et al., these primers could lead to a linear amplification of the 5′ LTR and of the 3′ LTR of the linear viral DNA (FIG. 2A3). The linear single-stranded DNA produced could hybridize to the homologous LTR sequences, which would result in an exponential amplification from the viral DNA and could consequently result in biases in the quantification of the 1-LTR bands.

The amplification with the primers 1LTR LA1 (5′-GCGCTTCAGCAAGCCGAGTCCT-3′, SEQ ID NO: 1) and 1LTR LA16 (5′-GTCACACCTCAGGTACCTTTAAGACCAATGAC-3′ SEQ ID NO: 2) was carried out with DNA of infected cells of the CEM cell line and the amplification product was cloned in the vector pGEMT-easy to give p1-LTR used as calibrator of the real-time PCR (FIG. 2B). In this framework, it was determined that the optimal duration of the elongation step of the PCR was 25 seconds. p2-LTR which comprises the LTRs in their entirety surrounding the gag and env regions is used as control (FIG. 2B). A quantitative PCR with the aid of the primers 1LTR LA1 and 1LTR LA16 and with p1-LTR as matrix leads to a sensitive (FIGS. 2C and 2D, Table 2) and efficient (200 copies/10⁶ cells) detection of the 1-LTR DNA. p2-LTR, subjected to the same protocol, leads to a weak amplification signal (FIGS. 2C and 2D, Table 2). In addition, p2-LTR was digested by ScaI to mimic the linear viral DNA found in the infected cells. The inventors have precisely quantified the purified DNA according to the protocol adapted to the total viral DNA. Amplifications starting from several dilutions of this DNA in non-infected cells leads to amplification signals which are negligible in comparison with the initial quantity of linear DNA (FIGS. 2C and 2D, Table 2), which confirms the amplification starting from linear viral DNA as shown by Yoder et al. (Yoder & Fishel (2006) Journal of Virological Methods 138, 1-2:201-6). The non-specific amplification starting from viral linear DNA was estimated at least of 1% with respect to the initial quantity. Globally, the amplification of P1-LTR with the primers and the protocol described here is representative of the quantification of the 1-LTR bands.

TABLE 2
amplifications obtained starting from the
plasmid 1-LTR, from the plasmid 2-LTR and from DNA
mimicking linear viral DNA after digestion of the
plasmid 2-LTR by the enzyme ScaI

| | Initial | Calculated | Percentage |
|---|---|---|---|
| | quantity | quantity | amplification |
| p1-LTR | 2 · 10⁵ | 1,925 · 10⁵ | 96.2 |
| | 2 · 10⁴ | 2,109 · 10⁴ | 100 |
| | 2 · 10³ | 2,002 · 10³ | 100 |
| p2-LTR | 2 · 10⁵ | 1,937 · 10³ | 0.7 |
| | 2 · 10⁴ | 2,242 · 10² | 0.8 |
| | 2 · 10³ | 5,311 · 10¹ | 1.9 |
| Linearized | 2 · 10⁵ | 6,733 · 101 | 0.03 |
| p2-LTR | 2 · 10⁴ | 9786 | 0.05 |
| | 2 · 10³ | ND | ND |

### Example 2

In order to validate the method described in Example 1, the inventors quantified the different forms of viral DNA during an infection of cells by the HIV-1 virus derived from pNL4-3: virus Δenv NLENG1-ES-IRES wild-type (WT) and NLENG1-ES-IRES D116N (Gelderblom et al. (2008) Retrovirology 9;5:60).

20 million CEM cells are infected by a virus pseudotyped by the envelope VSV-G (30 ng of antigen p24 per million of cell). 3 infection conditions are actualized: infection by a virus whose integrase is catalytically active (wild type, WT) in the absence or in the presence of 500 nM of raltegravir (RAL), an inhibitor of the integration reaction (Steigbigel et al. (2008) New England Journal of Medicine 359,4: 339-354). The last condition is infection by a virus whose integrase is catalytically inactive (D116N) (Shin et al. (1994), Journal of virology 68,3 :1633-1642).

The results expected are:
“WT”: “Wild-Type”: presence of mature DNA due to the activity of the integrase as well as the detection of integrated viral DNA.
“D116N”: Catalytically inactive integrase: absence of mature DNA and of integrated viral DNA due to the absence of integrase activity.
“RAL”: infection by a virus having a “WT” integrase in the presence of raltegravir: presence of mature viral DNA because the raltegravir is not an inhibitor of the maturation reaction (3′-processing). In contrast, an absence of integrated viral DNA is expected because the raltegravir is an inhibitor of the integration reaction.

1 hour after infection, the cells are washed in a phosphate buffer (PBS) and contacted with trypsin for 1 min at 37° C. The cells are then washed once in culture medium and twice in a large volume of PBS. The cells are then cultured in RPMI 1640 medium containing 10% of fetal calf serum. At each post-infection time a sample of 3 million cells is formed. In order to eliminate DNA used for the preparation of the virus, the cell pellet is subjected to digestion by 1500 U of DNaseI (Invitrogen, reference AM2238) in a buffer containing 20 mM Tris HCl pH 8.3, 50 mM KCl, 2 mM MgCl₂ for 10 min at ambient temperature. The cell pellet is then washed in PBS and the cells are centrifuged and then placed at −80° C. before use. The DNA is then extracted with the kit QIAamp DNA blood mini kit (Qiagen, reference 51104).

The total number of copies of viral DNA is determined by using primers (Butler et al. (2001) Nature Medicine 7:631-634) hybridizing in the region U5 of the LTR (MH531 primer: 5′-TGTGTGCCCGTCTGTTGTGT-3′, (SEQ ID NO: 29) and in the gag gene (MH532 primer:GAGTCCTGCGTCGAGAGAGC-3′, SEQ ID NO: 30) as well as the hybridization probes MH FL* (5′-CCCTCAGACCCTTTTAGTCAGTGTGGAAa-3′, SEQ ID NO: 31) and MH LC* (5′-TCTCTAGCAGTGGCGCCCGAACAGb, SEQ ID NO: 32) (a: fluorescein at the 3′ end; b: LC red 640 at the 5′ end and phosphorylation at the 3′ end) (Table 1).

The bands at 2-LTR are amplified by the primers flanking the LTR-LTR junction (HIVF and HIVR1 primers) (Brussel & Sonigo (2003) Journal of Virology 77:10119-10124). The viral DNA integrated is quantified by Alu-PCR (Brussel & Sonigo (2003) Journal of Virology 77:10119-10124). The U5-gag sequences, the 2-LTR junctions as well as the specific amplification of the integrated DNA are amplified in duplicate starting from 1/50 of the extraction of cell DNA. The number of copies is normalized per μg of DNA by measuring the quantity of β-globin gene in 1/50 of the DNA extract (Table 1).

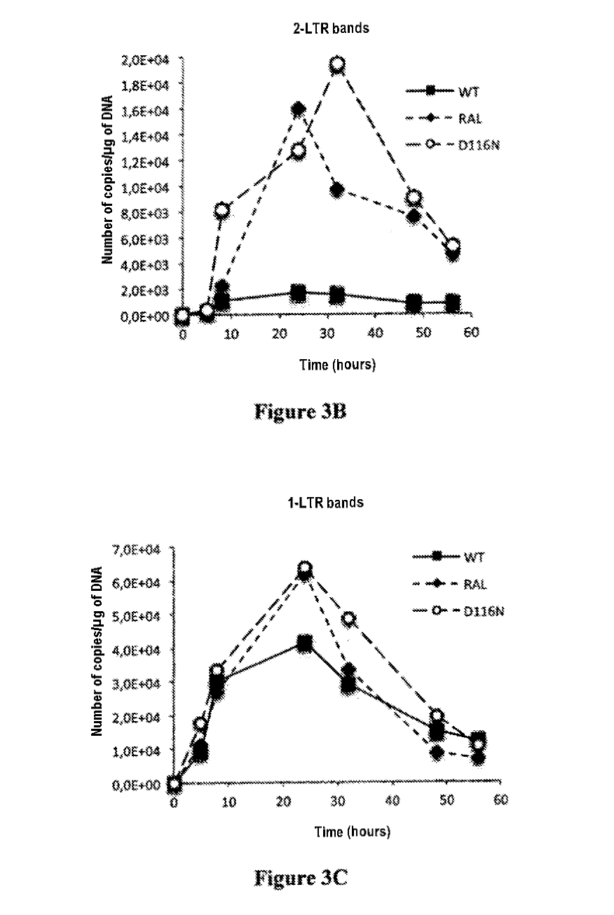

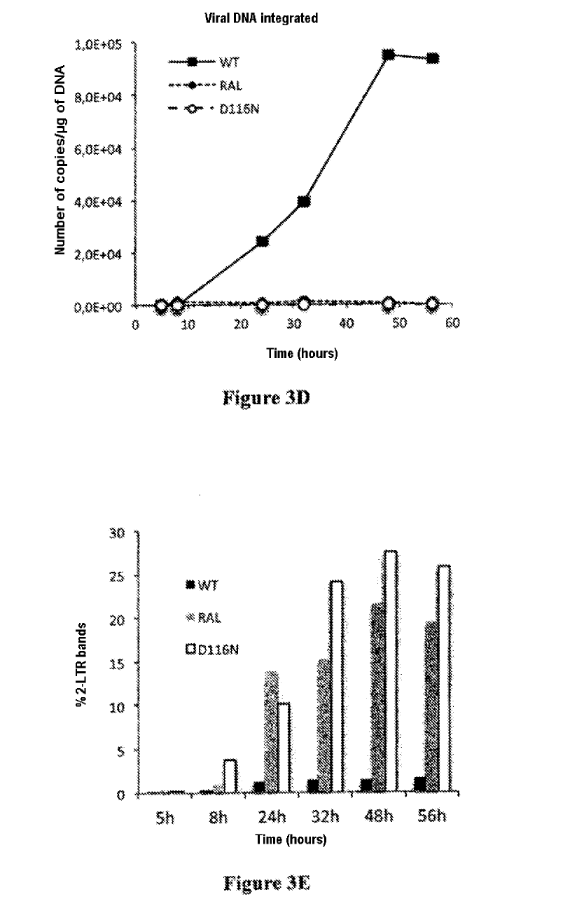

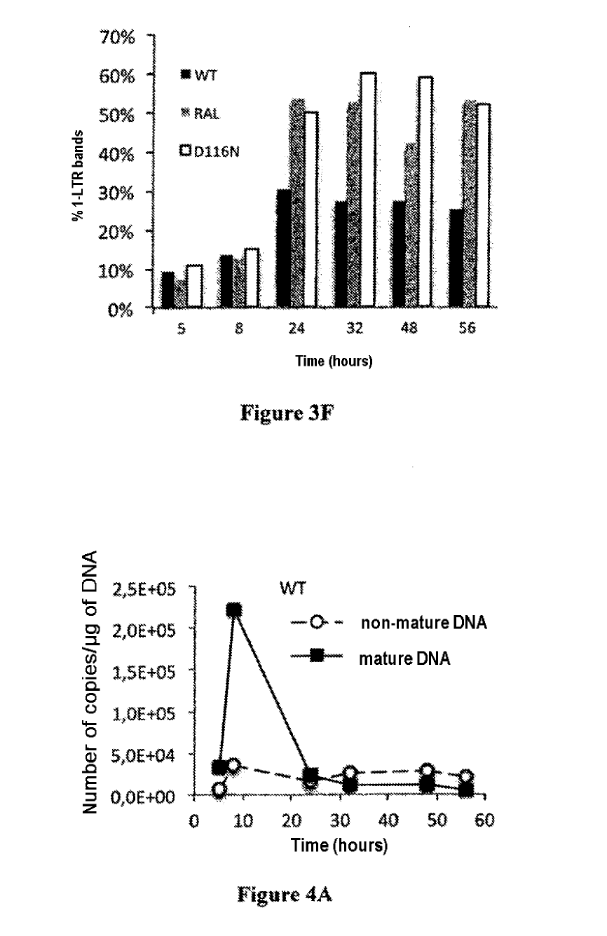

The results are presented in FIGS. 3A-3F. As these figures show, the synthesis of the total viral DNA, control of inverse transcription, is maximal 10 hours post-infection. The formation of 2-LTR bands is maximal 36 hours post-infection. The integration of the virus is complete 48 hours post-infection. These kinetics are in accordance with those described in the case of an infection by HIV-1 (Brussel & Sonigo (2003) Journal of Virology 77:10119-10124). The quantity of linear viral DNA (which comprises the two subpopulations: mature and non-mature) is achieved 8 hours post-infection. It is the same for the 3 conditions, i.e. “WT”, “RAL” and “D116N”, which is normal because neither the mutation D116N of the integrase, nor the raltegravir, influences the rate of conversion of RNA to linear DNA by the inverse transcriptase. The DNA is then degraded reflecting its degradation by the proteasome pathway (Butler et al. (2002) Journal of Virology 76:3739-47).

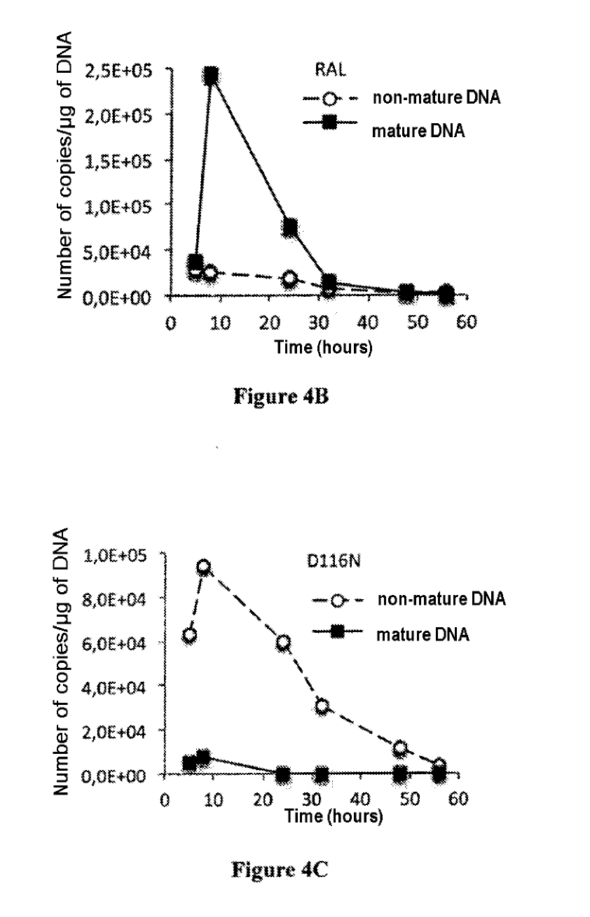

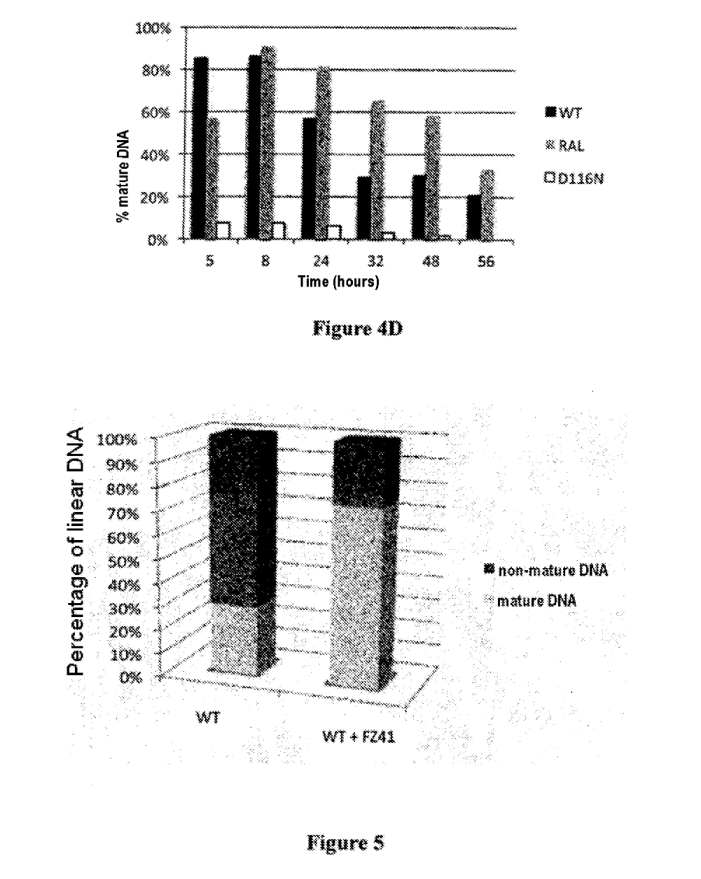

The discrimination between the mature and non-mature viral DNA was then validated following the protocol presented in Example 1. The results are presented in FIGS. 4A-4D.

Raltegravir, an integrase inhibitor, is an inhibitor of the integration reaction and not of the maturation of the ends in the context of an infection (Nguyen et al. (2011) Ann. N. Y. Acad Sci. 1222:83-9). The efficacy of this drug is proved in FIG. 3D (inhibition of the detection of integrated viral DNA, similar to the inhibition of the integration by a mutant of the catalytic site (D116N)). The quantity of mature viral DNA with or without raltegravir is similar (FIG. 4A-4B). In contrast, the mature viral DNA is quasi-undetectable in the case of an infection by the mutant of the catalytic site which has been shown as being totally devoid of activity including therefore for the catalysis of the maturation step (FIG. 4C). In addition, the percentage of linear viral DNA decreases as a function of the post-infection time with respect to non-mature DNA (FIG. 4D). Thus, this experiment underscores a less great stability of the mature viral DNA with respect to the immature linear viral DNA, the linear viral DNA being the viral form the least stable amongst the different viral genomes.

The styrylquinolines (SQ) are, in vitro, fixation inhibitors of integrase on viral DNA (Deprez et al. (2004) Mol. Pharmacol. 65:85-98) and thus represent a different class from that of raltegravir since the SQs act as competitive inhibitors of the maturation step. Although efficacious during a viral infection, the target step among these inhibitors has never been able to be clearly identified for lack of tool allowing, ex vivo, the step of maturation of the viral DNA to be characterized (Bonnenfant et al. (2004) Journal of Virology 78:5728-36). The method according to the invention has thus allowed it to be demonstrated that the styrylquinolines, of which the molecule FZ41 is the prototype, inhibit the maturation reaction of the viral DNA well.

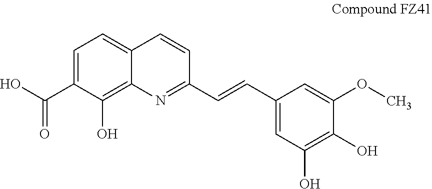 [SMILES: COc1cc(/C=C/c2ccc3ccc(C(=O)O)c(O)c3n2)cc(O)c1O]

In fact, CEM cells were infected by the virus having a catalytically active “wild-type” integrase. The cells were treated or untreated, at the time of infection, with 20 μM of FZ41. 20 hours post-infection the mature linear viral DNA and the immature viral DNA are quantified and reported FIG. 5. The results show an inhibition of the maturation of the viral DNA by the compound FZ41. In fact, the percentage of mature viral DNA is 75% in the absence of SQ (and consequently 25% of non-mature DNA) against 28% of mature DNA in the presence of FZ41. The inhibition of the maturation of the viral DNA is thus important in the presence of SQ.

The method according to the invention, reliable, sensitive, inexpensive, is thus well adapted to the screening of compounds inhibiting integrase with a view to testing their activity on the maturation of the linear DNA, the only substrate of the integration reaction.

Furthermore, this method can be declined in a clinical test with a view to a predictive test of avoidance of patients, the viral burden being associated with the quantity of viral DNA in the infected cells.

### Example 3

In order to evaluate the influence of the content of 2-LTR bands on the quantification of the 1-LTR bands according to the invention, Nalm-6 (ligase-4⁺) and Nalm-114 (ligase-4⁻) cells were infected with HIV-1 Δenv, either of the wild-type or of D116N type (catalytically inactive integrase). It has previously been shown that by Southern blotting that the ligase-4 is uniquely involved in the formation of the bands at 2-LTR (not in the formation of those at 1-LTR) and that the presence of the mutation D116N leads to a great accumulation of 2-LTR bands in a ligase-4⁺context (and in a lesser measure to a slight accumulation of 1-LTR bands) by the fact of a lack of integration. The results of the inventors confirm the strong inhibition (factor 40) of the formation of the 2-LTR bands at the same time for the wild-type of virus and D116N in Nalm-114 cells with respect to the Nalm-6 cells. The quantities of 1-LTR bands are similar for the cell lines infected by the virus of wild-type or of D116N type. These results confirm that the ligase-4 is not involved in the formation of 1-LTR bands. Importantly, the inventors have noticed that the quantity of 1-LTR bands was essentially unchanged whatever the level of accumulation of 2-LTR bands, which confirms that with the aid of the quantitative approach of the invention an exact quantification of 1-LTR bands in the cellular context and possible without bias is linked to the presence of 2-LTR bands.

### Example 4

Two other integrase inhibitors (INSTI), elvitegravir (EVG) and dolutegravir (DTG) were studied for their capacity to inhibit the maturation reaction (3′-processing) and were systematically compared to raltegravir (RAL). At the lowest concentrations of RAL, DTG and EVG completely inhibiting viral integration (500 nM) (the three inhibitors have similar IC₅₀s as far as the inhibition of viral integration is concerned, 2 nM for DTG and EVG, 8 nM for RAL), the maturation step was not affected whatever the inhibitor used. Interestingly, the increase in the concentration of inhibitor had different effects on this maturation reaction according to the compound. Thus, RAL remained essentially ineffective up to concentrations of 5 μM where 80% of the maturation activity was still visible. On the contrary, the maturation activity was greatly affected at 2.5 or 5 μM of DTG and EVG (maturation efficacy at approximately 40% and inferior to 25% at 2.5 μM and 5 μM respectively).

In fact, the in vitro studies with the aid of recombinant integrase show that the three inhibitors do not inhibit the maturation reaction at the same level. The IC50s relating to the maturation reaction are respectively 2 μM, 5 μM and 10 μM for DTG, EVG and RAL.

The different effects of each of the inhibitors on the maturation reaction were confirmed with the aid of a radiolabeled probe derived from the PCR. 500 nM of each inhibitor led to similar quantities of mature DNA, whereas at 5 1μM of inhibitor, DTG and EVG significantly inhibit maturation, on the contrary to RAL.

Interestingly, varying the concentrations of DTG and EVG from 500 nM to 5 μM increases the inhibition of maturation continuously but does not lead to an increase in the accumulation of 2-LTR or 1-LTR bands. In fact, at this range of concentrations where DTG and EVG completely inhibit integration, the 2 LTR bands represent almost 40% of the viral forms at 48 h post-infection. These data demonstrate that the accumulation of 2-LTR bands is principally connected with the inhibition of integration and not with the efficacy of the maturation reaction.

The efficacy of maturation was measured during the infection of primary CD4+ T lymphocytes with the aid of a Δenv virus of wild-type described previously (+/− RAL, EVG or DTG at 5 μM). It is observed that the efficacy of the maturation reaction is high (75%) but slightly slowed in the primary cells (the maturation maximum is achieved 24 h post-infection) and influenced by EVG and DTG (at 5 μM) but not by RAL. In addition, the accumulation kinetics of the 1-LTR and 2-LTR bands are similar in the primary cells with respect to cells of cellular line.

### Example 5

At the current time, the origin of the formation of 1-LTR bands does not remain very clear. Thus, for certain authors, the formation of 1-LTR bands requires a homologous recombination between 2 LTR of linear DNA in the nucleus although for others it involves an inverse transcription step in the cytoplasmic compartment.

A cell fractionation was carried out 24 h post-infection on MT4 cells infected with a Δenv virus carrying the mutation D116N described previously, that is to say when the nuclear import is accomplished, as shown by the maximum quantity of 2-LTR bands which are formed exclusively in the nucleus. In fact, the results of the inventors confirm that more than 99.5% of the 2-LTR bands are found in the nucleus. In comparison, the quantity of 1-LTR bands was much larger in the cytoplasmic fraction. In fact, in conditions where the nuclear import is maximal, the 1-LTR bands formed in the cytoplasm represent 10% of the total quantity of 1-LTR band. This is consistent with the kinetics of formation of 1-LTR bands where the 1-LTR bands represent 10% of the viral DNA from 5 h and 8 h post-infection and compatible with the hypothesis according to which at least one part of the 1-LTR bands could be formed during the inverse transcription step.

The peptides NLS-IN-Pen and SV40-NLS-Pen, which have been described as inhibiting the nuclear import of the pre-integration complex by the inhibition of the interaction between integrase and importine α (Levin et al. (2009) Retrovirology 6:112), were used in order to determine if the 1-LTR bands are formed uniquely during the inverse transcription step and do not necessitate the translocation of the pre-integration complex. HeLa cells, treated with one of the two peptides, were infected with a PnL4.3 virus in the presence of RAL. Without peptide, the 2-LTR bands accumulate to represent 19.3% of the total viral DNA at 48 h post-infection. Treatment with the peptides leads to an inhibition of the accumulation of 2-LTR bands (5.22% and 2.24 for NLS-IN-Pen and SV40-NLS-Pen respectively) underlining the inhibition of the nuclear import (3.7 and 8.6 times for NLS-IN-Pen and SV40-NLS-Pen respectively). Interestingly, under these conditions, the inventors observe a decrease in the formation of 1-LTR bands but less marked than for the inhibition relative to the 2-LTR bands (1.8 and 2.5 times for NLS-IN-Pen and SV40-NLS-Pen respectively).

It has likewise been possible to carry out the inhibition of the nuclear import of the pre-integration complex more specifically by mutations in the FLAP and/or CTS regions of the virus. Thus, HeLA cells were infected by a defective mutant, at the same time in the CTS and PPT regions. The quantities of 1-LTR and 2-LTR bands were normalized with respect to the quantities determined in wild-type conditions at 24 h post-infection. Although the inventors have found that the abrogation of the FLAP structure inhibits partially, and not completely, the cellular import of the pre-integration complex, the inventors have observed a substantial decrease in the quantity of 2-LTR bands (approximately 2 times). In this context, the inventors have demonstrated a concomitant decrease in the quantity of 1-LTR bands but to a lesser degree (1.4 times). Taken together, these data suggest clearly that the two mechanisms of formation of 1-LTR bands are not mutually exclusive: 1-LTR bands could be formed in the cytoplasm during the inverse transcription phase but the major part of the 1-LTR bands (90%) would be formed by homologous recombination, in the nucleus, after the translocation of the pre-integration complex.

TABLE 1

| Name of | | | | | |
|---|---|---|---|---|---|
| the probe | | SEQ | | | |
| or of the | | ID | | | |
| primer | Sequence | NO: | Target | Denaturation | PCR cycles |
| MH 531 | 5′-TGTGTGCCCGTCTGTTGTGT | 29 | Total DNA of | 95° C., 8 min | 95° C. 10s, 60° C. |
| MH 532 | 5′-GAGTCCTGCGTCGAGAGAGC | 30 | HIV-1 | | 10s, 72° C. 6s, |
| MH FL* | 5′-CCCTCAGACCCTTTTAGTCAGTGTGGAA^{a} | 31 | | | 50 cycles |
| MH LC* | 5′-TCTCTAGCAGTGGCGCCCGAACAG^{b} | 32 | | | |
| HIV F | 5′-GTGCCCGTCTGTTGTGTGACT | 33 | 2-LTR bands | 95° C., 8 min | 95° C. 10s, 66° C. |
| HIV R1 | 5′-ACTGGTACTAGCTTGTAGCACCATCCA | 34 | | | 10s, 72° C. 6s, |
| | | | | | 15 cycles then |
| HIV FL* | 5′-CCACACACAAGGCTACTTCCCTGA^{a} | 35 | | | 55 cycles with |
| HIV LC* | 5′-TGGCAGAACTACACACCAGGGC^{b} | 36 | | | a lowering of |
| | | | | | the |
| | | | | | hybridization |
| | | | | | temperature of |
| | | | | | 0.5° C./cycle to |
| | | | | | 59° C. |
| L-M667 | 5′-ATGCCACGTAAGCGAAACTCTGGCTAACTA | | Integrated DNA | 95° C., 8 min | 95° C. 10s, 60° C. |
| Alu1 | GGGAACCCACTG | 37 | of HIV-1 | | 10s, 72° C. 170s, |
| Alu2 | 5′-TCCCAGCTACTGGGGAGGCTGAGG | 38 | (first round | | 12 cycles |
| | 5′-GCCTCCCAAAGTGCTGGGATTACAG | 39 | of PCR) | | |
| Lambda | 5′-TATGCCACGTAAGCGAAACT | 40 | Integrated DNA | 95° C., 8 min | 95° C. 10s, 60° C. |
| AA55M | 5′-GCTAGAGATTTTCCACACTGACTAA | 41 | of HIV-1 | | 10s, 72° C. 9s, |
| LTR FL* | 5′-CACAACAGACGGGCACACACTACTTGA^{a} | 3 | (second round | | 50 cycles |
| LTR LC* | 5′-CACTCAAGGCAAGCTTTATTGAGGC^{b} | 4 | of PCR) | | |
| 32t | 5′-GCGCGCGGCGGTGACCCGGGAGATCTGAATTC | 25 | Linear DNA of | 95° C., 8 min | 95° C. 10s, 60° C. |
| MS1 | 5′-CTCGCCTCTTGCCGTGCGCG | 9 | HIV-1 (first | | 10s, 72° C. 31s, |
| | | | round of PCR) | | 12 cycles |
| 25t | 5′-GCGGTGACCCGGGAGATCTGAATTC | 5 | Linear DNA of | 95° C., 8 min | 95° C. 10s, 60° C. |
| MS2 | 5′-GAGTCCTGCGTCGAGAGATC | 26 | HIV-1 (2^{nd} | | 10s, 72° C. 26s, |
| MH FL* | 5′-CCCTCAGACCCTTTTAGTCAGTGTGGAA | 27 | round of PCR) | | 50 cycles |
| MH LC* | 5′-TCTCTAGCAGTGGCGCCCGAACAG^{b} | 28 | | | |
| 1LTR LA1 | 5′-GCGCTTCAGCAAGCCGAGTCCT | 1 | 1-LTR bands | 95° C., 8 min | 95° C. 10s, 60° C. |
| 1LTR LA16 | 5′-GTCACACCTCAGGTACCTTTAAGACCAATGAC | 2 | | | 10s, 72° C. 25s, |
| LTR FL* | 5′-CACAACAGACGGGCACACACTACTTGA^{a} | 3 | | | for 50 cycles |
| LTR FC* | 5′-CACTCAAGGCAAGCTTTATTGAGG^{b} | 4 | | | |

^{a}Modified by fluorescein in 3′.
^{b}Modified by the fluorophore LC red 640 in 5′phosphorylated in 3′.
The primers and the probes were obtained from TIB MOLBIOL (Berlin, Germany).
*probe

1. A method of quantification of linear forms of the DNA genome of the human immunodeficiency virus 1 (HIV-1) in a sample, in which:
a ligation reaction is carried out with a double-stranded oligonucleotide adapted for a ligation with the 3′ mature linear form and/or the the 3′ non-mature linear form of the DNA genome of HIV-1,
a quantitative PCR is carried out starting from the ligation product obtained in the preceding step, and
the quantity of the 3′ mature linear form and/or of the 3′ non-mature linear form of the DNA genome of the HIV-1 in the sample is deduced therefrom.

2. The method as claimed in claim 1, in which the linking oligonucleotide allows a ligation with the 3′ mature linear form and the 3′ non-mature linear form of the linear form of the DNA genome of the HIV-1 and comprises a sticky end of two nucleotides of sequence 5′-GT-3′, and in which the total quantity of the linear forms of the genome of the HIV-1 in the sample is deduced from the quantitative PCR.

3. The method as claimed in claim 1, in which the linking oligonucleotide allows a ligation specifically with the 3′ non-mature linear form of the DNA genome of the HIV-1 and comprises a blunt end, and in which the quantity of the non-mature linear form of the DNA genome of the HIV-1 in the sample is deduced from the quantitative PCR.

4. The method as claimed in claim 1, in which the quantity of the mature linear form of the DNA genome of the HIV-1 in the sample is deduced by subtracting the quantity of the non-mature linear form of the DNA genome of the HIV-1 in the sample quantified according to claim 3 from the total quantity of the linear forms of the genome of the HIV-1 in the sample quantified according to claim 2.

5. The method as claimed in claim 1, in which the quantitative PCR is carried out in two steps: a first amplification step without quantification of the nucleic acid amplified and a second amplification step with quantification of the nucleic acid amplified.

6. The method as claimed in claim 5, in which the first step of the quantitative PCR comprises from 9 to 30 cycles.

7. The method as claimed in claim 5, in which the first step of the quantitative PCR comprises from 10 to 14 cycles.

8. The method as claimed in claim 1, in which the quantitative PCR is a real-time PCR.

9. The method as claimed in claim 8, in which the real-time PCR is conducted with hybridization probes.

10. A method of quantification of the 1-LTR circular form of the genome of the HIV-1 in a sample in which a quantitative PCR is carried out with the aid of two primers comprising respectively a sequence 5′-GCGCTTCAGCAAGCCGAGTCCT-3′ (SEQ ID NO: 1), or a sequence having at least 90% identity with SEQ ID NO: 1, and a sequence 5′-GTCACACCTCAGGTACCTTTAAGACCAATGAC-3′ (SEQ ID NO: 2), or a sequence having at least 90% identity with SEQ ID NO: 2.

11. A method of screening for compounds capable of modifying the quantity of a non-integrated form of the genome of the HIV-1, comprising the implementation of a method as defined claim 1.

12. The method of screening for compounds capable of modifying the quantity of a non-integrated form of the genome of the HIV-1, in which the compounds are capable of inhibiting the maturation of the linear form of the DNA genome of the HIV-1 and a method is implemented as defined in claim 1.

13. A method of screening for compounds capable of modifying the quantity of a non-integrated form of the genome of the HIV-1, comprising the implementation of a method as defined in claim 10.


SEQUENCE LISTING (41 sequences)

SEQ ID NO: 1 - DNA, 22 bp, Artificial Sequence
  Description of Artificial Sequence Synthetic PCR primer
gcgcttcagcaagccgagtcct

SEQ ID NO: 2 - DNA, 32 bp, Artificial Sequence
  Description of Artificial Sequence Synthetic PCR primer
gtcacacctcaggtacctttaagaccaatgac

SEQ ID NO: 3 - DNA, 27 bp, Artificial Sequence
  Description of Artificial Sequence Synthetic Hybridation probe
cacaacagacgggcacacactacttga

SEQ ID NO: 4 - DNA, 25 bp, Artificial Sequence
  Description of Artificial Sequence Synthetic Hybridation probe
cactcaaggcaagctttattgaggc

SEQ ID NO: 5 - DNA, 25 bp, Artificial Sequence
  Description of Artificial Sequence Synthetic Oligonucleotide linker
gcggtgacccgggagatctgaattc

SEQ ID NO: 6 - DNA, 13 bp, Artificial Sequence
  Description of Artificial Sequence Synthetic Oligonucleotide linker
gtgaattcagatc

SEQ ID NO: 7 - DNA, 11 bp, Artificial Sequence
  Description of Artificial Sequence Synthetic Oligonucleotide linker
gaattcagatc

SEQ ID NO: 8 - DNA, 51 bp, Artificial Sequence
  Description of Artificial Sequence Synthetic PCR primer
gcggtgacccgggagatctgaattcagtactggaagggctaatttggtccc

SEQ ID NO: 9 - DNA, 20 bp, Artificial Sequence
  Description of Artificial Sequence Synthetic PCR primer
ctcgcctcttgccgtgcgcg

SEQ ID NO: 10 - DNA, 6 bp, Artificial Sequence
  Description of Artificial Sequence Synthetic Restriction site
agtact

SEQ ID NO: 11 - DNA, 6 bp, Artificial Sequence
  Description of Artificial Sequence Synthetic Mutated Restriction site
agaact

SEQ ID NO: 12 - DNA, 43 bp, Artificial Sequence
  Description of Artificial Sequence Synthetic PCR primer
cccgagagctgcatccggagaactacaaagactgctgacatcg

SEQ ID NO: 13 - DNA, 43 bp, Artificial Sequence
  Description of Artificial Sequence Synthetic 14
cgatgtcagcagtctttgtagttctccggatgcagctctcggg

SEQ ID NO: 14 - DNA, 6 bp, Artificial Sequence
  Description of Artificial Sequence Synthetic Restriction site
catatg

SEQ ID NO: 15 - DNA, 44 bp, Artificial Sequence
  Description of Artificial Sequence Synthetic PCR primer
ccgggagatctgaattcagtcatatggaagggctaatttggtcc

SEQ ID NO: 16 - DNA, 44 bp, Artificial Sequence
  Description of Artificial Sequence Synthetic PCR primer
ggaccaaattagcccttccatatgactgaattcagatctcccgg

SEQ ID NO: 17 - DNA, 50 bp, Artificial Sequence
  Description of Artificial Sequence Synthetic PCR primer
cgggagatctgaattcagtactgctagagattttccacactgactaaaag

SEQ ID NO: 18 - DNA, 6 bp, Artificial Sequence
  Description of Artificial Sequence Synthetic Mutated Restriction site
agcact

SEQ ID NO: 19 - DNA, 30 bp, Artificial Sequence
  Description of Artificial Sequence Synthetic PCR primer
agctgcatccggagcacttcaagaactgct

SEQ ID NO: 20 - DNA, 30 bp, Artificial Sequence
  Description of Artificial Sequence Synthetic PCR primer
agcagttcttgaagtactccggatgcagct

SEQ ID NO: 21 - DNA, 6 bp, Artificial Sequence
  Description of Artificial Sequence Synthetic Restriction site muti
aatact

SEQ ID NO: 22 - DNA, 34 bp, Artificial Sequence
  Description of Artificial Sequence Synthetic PCR primer
gtgactggtgaatactcaaccaagtcattctgag

SEQ ID NO: 23 - DNA, 34 bp, Artificial Sequence
  Description of Artificial Sequence Synthetic PCR primer
ctcagaatgacttggttgagtattcaccagtcac

SEQ ID NO: 24 - DNA, 13 bp, Artificial Sequence
  Description of Artificial Sequence Synthetic Oligonucleotide linker
tagaattcagatc

SEQ ID NO: 25 - DNA, 32 bp, Artificial Sequence
  Description of Artificial Sequence Synthetic PCR primer
gcgcgcggcggtgacccgggagatctgaattc

SEQ ID NO: 26 - DNA, 20 bp, Artificial Sequence
  Description of Artificial Sequence Synthetic 27 oligonucleotide
gagtcctgcgtcgagagatc

SEQ ID NO: 27 - DNA, 28 bp, Artificial Sequence
  Description of Artificial Sequence Synthetic Hybridation probe
ccctcagacccttttagtcagtgtggaa

SEQ ID NO: 28 - DNA, 24 bp, Artificial Sequence
  Description of Artificial Sequence Synthetic Hybridation probe
tctctagcagtggcgcccgaacag

SEQ ID NO: 29 - DNA, 20 bp, Artificial Sequence
  Description of Artificial Sequence Synthetic PCR primer
tgtgtgcccgtctgttgtgt

SEQ ID NO: 30 - DNA, 20 bp, Artificial Sequence
  Description of Artificial Sequence Synthetic PCR primer
gagtcctgcgtcgagagagc

SEQ ID NO: 31 - DNA, 28 bp, Artificial Sequence
  Description of Artificial Sequence Synthetic Hybridation probe
ccctcagacccttttagtcagtgtggaa

SEQ ID NO: 32 - DNA, 24 bp, Artificial Sequence
  Description of Artificial Sequence Synthetic Hybridation probe
tctctagcagtggcgcccgaacag

SEQ ID NO: 33 - DNA, 21 bp, Artificial Sequence
  Description of Artificial Sequence Synthetic PCR primer
gtgcccgtctgttgtgtgact

SEQ ID NO: 34 - DNA, 27 bp, Artificial Sequence
  Description of Artificial Sequence Synthetic PCR primer
actggtactagcttgtagcaccatcca

SEQ ID NO: 35 - DNA, 24 bp, Artificial Sequence
  Description of Artificial Sequence Synthetic oligonucleotide Hybridation probe
ccacacacaaggctacttccctga

SEQ ID NO: 36 - DNA, 22 bp, Artificial Sequence
  Description of Artificial Sequence Synthetic Hybridation probe
tggcagaactacacaccagggc

SEQ ID NO: 37 - DNA, 42 bp, Artificial Sequence
  Description of Artificial Sequence Synthetic PCR primer
atgccacgtaagcgaaactctggctaactagggaacccactg

SEQ ID NO: 38 - DNA, 24 bp, Artificial Sequence
  Description of Artificial Sequence Synthetic PCR primer
tcccagctactggggaggctgagg

SEQ ID NO: 39 - DNA, 25 bp, Artificial Sequence
  Description of Artificial Sequence Synthetic PCR primer
gcctcccaaagtgctgggattacag

SEQ ID NO: 40 - DNA, 20 bp, Artificial Sequence
  Description of Artificial Sequence Synthetic PCR primer
tatgccacgtaagcgaaact

SEQ ID NO: 41 - DNA, 25 bp, Artificial Sequence
  Description of Artificial Sequence Synthetic PCR primer
gctagagattttccacactgactaa